(19) United States
(12) Patent Application Publication
(10) Pub. No.: US 2025/0322657 A1
(43) Pub. Date: Oct. 16, 2025

(54) Using Device Sensors to Guide Scent Dispenser Installation

(71) Applicant: Pura Scents, Inc., Pleasant Grove, UT (US)

(72) Inventors: Trevor Davis, American Fork, UT (US); Róbert Duriancik, Pleasant Grove, UT (US)

(21) Appl. No.: 19/054,886

(22) Filed: Feb. 16, 2025

Related U.S. Application Data
(60) Provisional application No. 63/554,487, filed on Feb. 16, 2024.

(51) Int. Cl. G06V 20/00; G06T 7/62; G06T 7/73

(52) U.S. Cl. CPC G06V 20/36; G06T 7/62; G06T 7/74

(57) ABSTRACT

A method of using device sensors to install a scent dispensing device, the method including receiving a request from a sensor mapping application that a scent dispensing device is being installed in a space, capturing using a device sensor on a device a space mapping of at least a portion of the space where the scent dispensing device is being installed, determining one or more installation locations within the space mapping, determining an optimal scent dispensing location from the one or more installation locations within the space mapping, generating a graphical representation depicting an installation of the scent dispensing device at the optimal scent dispensing location, and presenting in a user interface on a display screen of the device a graphical representation depicting the installation of the scent dispensing device at the optimal scent dispensing location.

### BACKGROUND

The present disclosure relates to using device sensors to guide scent dispenser installation.

Existing solutions for dispensing scents within a room include liquid scented oil diffusers, wax or cork disks infused with scented oils, or nebulizers. The scented oil can be dispersed throughout the room by heating elements and/or fans. To emit the scents in the rooms, the heating elements and/or fans are turned on to emit a scent and turned off to stop scent diffusion. Existing solutions provide instructions for a user to install a scent dispenser by plugging the scent dispenser into an available power outlet. In some existing solutions, the installation instructions also provide notes on ideal installation positions that a user can read and then manually install the scent dispenser based on the installation instructions.

### SUMMARY

In some aspects, the techniques described herein relate to a method of using device sensors to install a scent dispensing device, including: receiving a request from a sensor mapping application that a scent dispensing device is being installed in a space; capturing using a device sensor on a device a space mapping of at least a portion of the space where the scent dispensing device is being installed; determining one or more installation locations within the space mapping; determining an optimal scent dispensing location from the one or more installation locations within the space mapping; generating a graphical representation depicting an installation of the scent dispensing device at the optimal scent dispensing location; and presenting in a user interface on a display screen of the device the graphical representation depicting the installation of the scent dispensing device at the optimal scent dispensing location.

In some aspects, the techniques described herein relate to a method, wherein capturing the space mapping further includes: determining based on the space mapping one or more dimensions of the space.

In some aspects, the techniques described herein relate to a method, wherein the dimensions of the space includes one or more of a length of the space, a width of the space, a height of the space, a volume of the space, and an area of the space.

In some aspects, the techniques described herein relate to a method, wherein the device sensor is a camera of a client device and the space mapping is one or more images of the space captured by the camera.

In some aspects, the techniques described herein relate to a method, wherein determining the installation locations within the space mapping further includes performing object recognition to determine one or more objects present within the one or more images.

In some aspects, the techniques described herein relate to a method, wherein the one or more determined objects present within the images may be compared to a database of common objects to determine one or more dimensions of the space.

In some aspects, the techniques described herein relate to a method, wherein determining the installation locations within the space mapping further includes determining one or more power outlet locations that the scent dispensing device can plug into.

In some aspects, the techniques described herein relate to a method, wherein determining the optimal scent dispensing location from the one or more installation locations within the space mapping further includes: determining an obstruction present within the space mapping; determining a walkway present within the space mapping; and identifying which of the power outlet locations is optimized based on the determined obstruction and the determined walkway.

In some aspects, the techniques described herein relate to a method, wherein the graphical representation depicting the installation of the scent dispensing device at the optimal scent dispensing location is an overlay and presenting in the user interface on the display screen of the device further includes placing the overlay over the space mapping on the display screen of the device.

In some aspects, the techniques described herein relate to a method, wherein determining one or more installation locations within the space mapping further includes: detecting a scent dispensing device within the space mapping; estimating space dimensions using a known scale of the scent dispensing device; and determining the one or more installation locations based on the estimated space dimensions.

In some aspects, the techniques described herein relate to a scent dispensing device installation system including: a scent dispensing device configured to be installed in a space; a device sensor configured to capture a space mapping of the space; and a sensor mapping application configured to: receive the space mapping from the device sensor; determine an installation location for the scent dispensing device from the space mapping; optimize the installation location based on other context in the space mapping; and causing a user interface on a display screen to present a graphical representation depicting the installation of the scent dispensing device at the optimal scent dispensing location.

In some aspects, the techniques described herein relate to a scent dispensing device installation system, wherein when the sensor mapping application receives the space mapping from the device sensor, the sensor mapping application is further configured to determine a dimension of the space based on the space mapping.

In some aspects, the techniques described herein relate to a scent dispensing device installation system, wherein the dimension of the space includes one or more of a length of the space, a width of the space, a height of the space, a volume of the space, and an area of the space.

In some aspects, the techniques described herein relate to a scent dispensing device installation system, wherein the sensor mapping application is further configured to identify an object in the space mapping using image recognition.

In some aspects, the techniques described herein relate to a scent dispensing device installation system, wherein the object in the space mapping is the scent dispensing device and wherein determining the dimensions of the space further includes using a known scale of the scent dispensing device to estimate the dimensions of the space.

In some aspects, the techniques described herein relate to a scent dispensing device installation system, wherein the sensor device is a camera of a client device and the space mapping is one or more images of the space captured by the camera.

In some aspects, the techniques described herein relate to a scent dispensing device installation system, wherein the sensor device is a lidar sensor that maps out a plurality of distances to capture the space mapping.

In some aspects, the techniques described herein relate to a scent dispensing device installation system, wherein scent dispensing device includes an audio output device and the sensor device includes an audio input device configured to capture a sound emitted from the audio output device and determine a distance between the scent dispensing device and the audio input device to capture the space mapping.

In some aspects, the techniques described herein relate to a method of guiding installation of a scent dispensing device, the method including: capturing, using a device sensor, a space mapping of a space in which a scent dispensing device is being installed; identifying, using a processor of a sensor mapping application, one or more objects within the space mapping, the one or more objects including the scent dispensing device, a power outlet, and a blocking object; determining, using the processor of the sensor mapping application, one or more dimensions of the space using the space mapping; determining, using the processor of the sensor mapping application, a first relative positions in the space mapping of the power outlet and a second relative position in the space mapping of the blocking object based on the one or more dimensions of the space mapping; determining, using the processor of the sensor mapping application, an optimal scent dispensing location based on the first relative positions in the space mapping of the power outlet and the second relative position in the space mapping of the blocking object; and presenting for display in a graphical user interface, an installation instruction to install the scent dispensing device at the optimal scent dispensing location.

In some aspects, the techniques described herein relate to a method, wherein the one or more dimensions of the space include a length of the space, a width of the space, a height of the space, a volume of the space, and an area of the space.

However, this list of features and advantages is not all-inclusive, and many additional features and advantages are within the scope of the present disclosure. Moreover, it should be noted that the language used in the present disclosure has been principally selected for readability and instructional purposes, and not to limit the scope of the subject matter disclosed herein.

### BRIEF DESCRIPTION OF THE DRAWINGS

FIG. 1 is a block diagram illustrating an example system for using device sensors to guide scent dispenser installation.

FIG. 2 is a block diagram illustrating a client device for using device sensors to guide scent dispenser installation.

FIGS. 3A and 3B depict a graphical representation of using device sensors to guide scent dispenser installation.

FIG. 4 depicts a graphical representation of using a client device to determine scent dispenser installation.

FIG. 5 is a flowchart of an example method of using device sensors to guide scent dispenser installation.

FIG. 6 is a graphical representation of using a sound wave to determine scent dispenser installation.

FIG. 7 is a graphical representation of using images to determine scent dispenser installation.

### DETAILED DESCRIPTION

The technology described in this disclosure relates to using device sensors to guide scent dispenser installation. As an example, the technology allows for a device sensors on a client device, such as a phone to map out a space and determine one or more optimal scent dispenser locations. By using the built-in device sensors inside of a user's phone or other device, the user can quickly use the device sensors to map out a space during installation without any additional tools and then a sensor mapping application is able to use the device sensors to analyze the captured data and determine the optimal or best place to position the scent dispenser within the space.

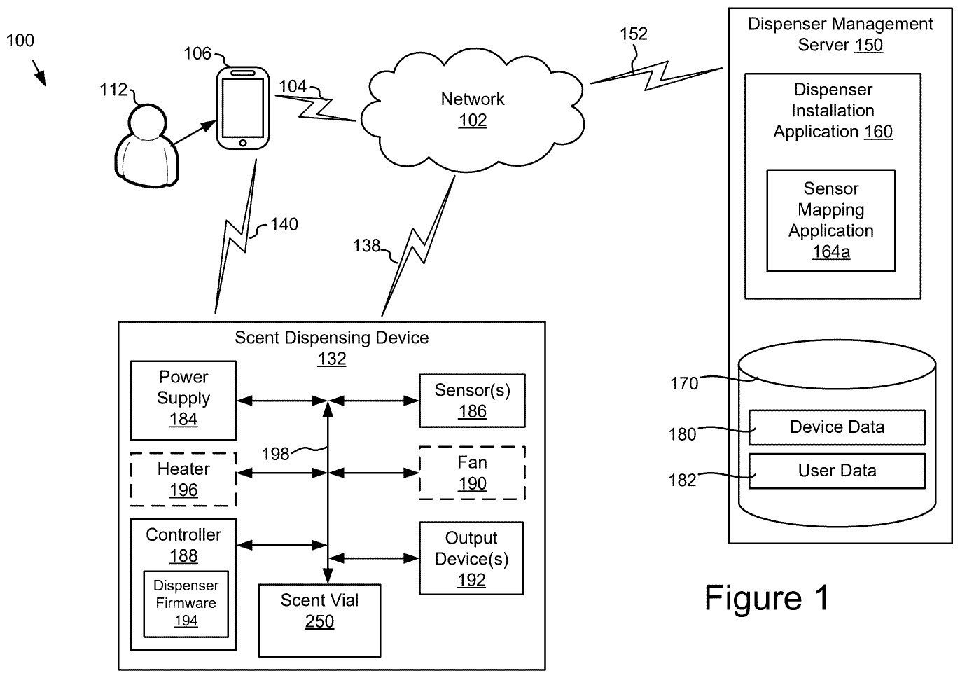

FIG. 1 is a block diagram illustrating an example system 100 for using device sensors to guide scent dispenser installation. The system 100 may include one or more scent dispensing devices 132 that can be positioned in an area, such as a room, space, vehicle, etc. In a typical implementation, a single scent dispensing device 132 may be being installed and positioned within a space (such as a room in a house, etc.), however in large spaces multiple scent dispensing devices 132 may be installed together throughout the space for effective scent dispensing where the devices 132 may work together and the sensor mapping application 164 may take into effect the locations of where other scent dispensing devices 132 are installed when determining optimal scent dispenser locations. In some implementations, the scent dispensing device 132 may be configured to plug into a wall outlet to provide power to the scent dispensing device 132. In other implementations, the scent dispensing device 132 may be battery powered, such as a car device or other portable device that is positioned within a vehicle or other space.

As shown, the illustrated system 100 further includes client device(s) 106 and a server 150, which are electronically communicatively coupled via a network 102 for interaction with one another and the scent dispenser(s) 132, etc., using standard networking protocols, as reflected by signal lines 104, 138, and 152. In some implementations, the scent dispensing device 132 may instead be communicatively coupled to the client device(s) 106, such as by Bluetooth, beacons, or other networking protocols (as reflected by signal line 140) in order to allow the scent dispensing device 132 to connect with a paired client device(s) 106 when in proximity rather than through the network 102.

The dispenser installation application 160 operable by the dispenser management server 150 can receive operational data from the scent dispensing device 132 in association with the device 106 and/or user(s) 112 with which they are associated. The dispenser installation application 160 can receive installation requests for a scent dispensing device 132, such as when a scent dispensing device 132 is first activated or when a client device 106 begins running an application that guides the installation process.

The dispenser installation application 160 can include a sensor mapping application 164. The sensor mapping application 164 may include software and/or logic to use device sensors on a client device 106 to capture information about a space and determine a mapping of the space. The sensor mapping application 164 can use the mapping of the space to determine one more scent dispenser device 132 installation locations. In some implementations, the sensor mapping application 164 may use one or more internal or third party artificial intelligence applications to determine answers to queries, such as estimated sizes of the various spaces, determinations on optimal device placement, determinations of various air flow properties, etc. These artificial intelligence applications may include a large data set or knowledge base and may use various processors to make inferred determinations based on the training sets and large data sets accessible to the artificial intelligence applications.

The dispenser management server 150 includes a data store 170 storing various types of data used by the dispenser installation application 160. Example data types include device data 180 and user data 182. The device data 180 may include a device model, a scent vial type, usage statistics, scent diffusion time, temperature variations, etc. The user data 182 may include entries for the users 112 of the system 100. A given entry may include a unique identifier for the user, a unique identifier for the user device 106, contact information for the user (e.g., address, phone number, electronic address (e.g., email)), payment information, scent subscription information specifying which reoccurring scent vials 250 may be shipped to the user, etc.

An example scent dispenser device 132 is depicted as including a power supply 184, one or more sensor(s) 186, a controller 188, output device(s) 192, dispenser firmware 194, a fan 190, a heater 196, and any number of scent vial(s) 250. The components 184, 186, 188, 190, 192, 196, and 250 are communicatively coupled via a communications bus 198. The controller 188 may include a non-transitory memory device (e.g., a non-volatile memory device), or may be coupled to a non-transitory memory device also coupled for communication via the bus 198. The non-transitory memory device may store software and/or firmware that specially configures the controller, such as the dispenser firmware 194. The power supply 184 may be any AC and/or DC power supply for powering the scent dispensing device 132. In some implementations, the power supply 184 may be battery powered and may be configured to charge when plugged into an AC and/or DC power supply or positioned on a wireless charging base. The controller 188 may be a microchip that controls the constituent electronics (e.g., sensor(s) 186, output device(s) 192, fan 190, heater 196, etc.) of the scent dispensing device 132.

The one or more sensor(s) 186 may include one or more temperature sensors for detecting the ambient temperature adjacent to the scent dispensing device 132. In some implementations, the one or more sensors(s) 186 may include a humidity to determine efficient diffusion of the scent based on various humidity readings. In some implementations, the one or more sensor(s) 186 may include a vial sensor for sensing when to replace a scent vial(s) 250 installed in the scent dispensing device 132, optical or other sensor(s) or electronics for detecting an identity of scent vial(s) 250 installed in the scent dispensing device 132, ambient light sensor to detect a light level in a surrounding environment, and/or a motion sensor to detect motion in the surrounding environment, etc. In some embodiments, the sensors may include an audio sensor to detect audio sounds in the surrounding environment. In some embodiments, the sensors 186 may include a separate temperature sensor for the scent vial 250 (e.g., to measure temperatures at the scent vial 250, which can be used to adjust a speed and/or frequency of the fan 190).

The sensors 186 may include a transceiver having a wireless interface configured to communicate with the devices coupled to the network 102, such as the dispenser management server 150, and/or other components of the network 102 using standard communication protocols, such as Internet protocols. Further, the transceiver may be configured to wirelessly transmit data via a network to connect to other devices, such as the mobile device 106. By way of further example, the transceiver may transmit data to the mobile device 106 to which it is linked using a protocol compliant with IEEE 802.15, such as Zigbee®, Z-Wave®, Bluetooth®, or another suitable standard. Further embodiments are also possible and contemplated. In some embodiments, the transceiver may be embedded in the controller 188 or may be a component distinct from the controller and coupled to the controller 188 via the bus 198.

The output device(s) 192 may include light sources and/or audio reproduction devices, although further suitable output devices are also contemplated and applicable. In some implementations, the light sources and/or audio reproduction devices may be controlled to produce output consistent with a scent being emitted by the scent dispenser (e.g., a low, soothing light and music may be output in conjunction with a relaxing scent being emitted), or to communicate various alerts, such as low power, low scent vial levels, etc. In some implementations, the output device(s) 192 may correlate with a scent dispensing output.

In some implementations, the scent dispensing device 132 may include the fan 190. The fan 190 may include a motor that has one or more fan blades that force air through the device 132 when the motor is operating. The fan 190 may operate at various speeds based on how quickly or slowly the motor runs. The fan 190 may be configured to nest within a housing of the device 132 and cause airflow to move through the device and across a scent vial 250 for scent diffusion. In some implementations, the fan 190 may be able to turn on and off as signaled by the microcontroller 188 which results in substantially immediate scent diffusion. In some implementations the fan 190 and the motor speed may be associated with scent settings and based on the level of the motor operating the fan 190 different scent diffusions can be released to fill different areas over different times based on the scent settings.

The scent vial 250 may be removable and contain a liquid scenting agent that diffuses a scent into the nearby air. The scent vial 250 may be replaceable when the scent is diminished and a new scent vial 250 can be inserted into the device 132. The scent vials 250 may have various scent profiles and information about the scent vials 250 may be stored in the dispenser installation application 160 to store the various scent profiles, ages of the scent vials 250, duration of use of the scent vials 250, exposed temperatures of the scent vials 250, etc.

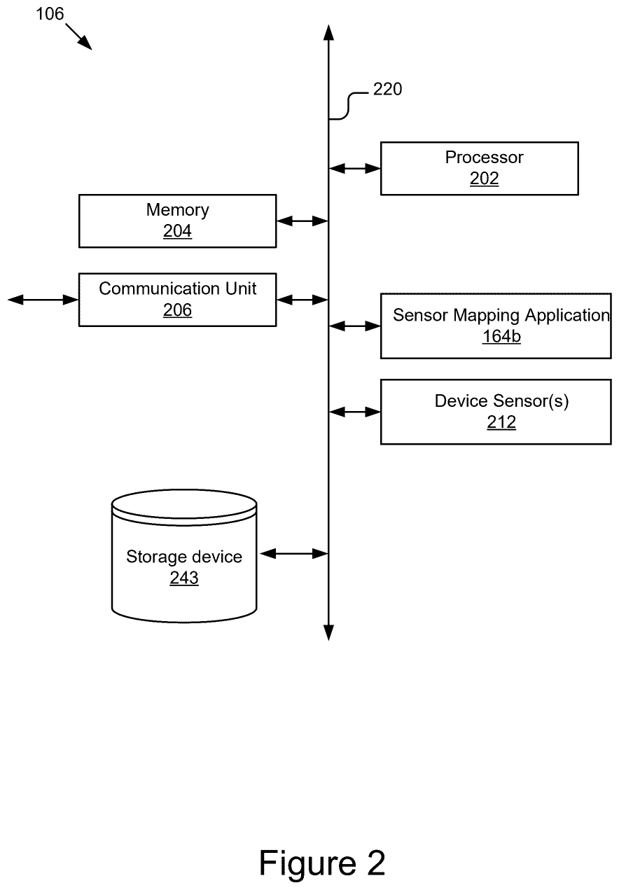

The client device(s) 106 (also referred to individually and collectively as 106) are computing devices having data processing and communication capabilities. In some embodiments, a client device 106 may include, such as the example shown in more detail in FIG. 2, a processor (e.g., virtual, physical, etc.), a memory, a power source, a network interface, and/or other software and/or hardware components, such as a display, graphics processor, wireless transceivers, keyboard, camera, sensors, firmware, operating systems, drivers, various physical connection interfaces (e.g., USB, HDMI, etc.).

The client devices 106 may couple to and communicate with one another and the other entities of the system 100 via the network 102 using a wireless and/or wired connection. Examples of client devices 106 may include, but are not limited to, mobile phones (e.g., feature phones, smart phones, etc.), tablets, smartwatches or other smart wearables, laptops, desktops, netbooks, server appliances, servers, virtual machines, TVs, set-top boxes, media streaming devices, portable media players, navigation devices, personal digital assistants, car access panels, etc. In addition, while a single client device 106 is depicted in FIG. 1, it should be understood that any number of client devices 106 may be included.

As shown, the client device 106 may include a scent application, which allows the user to set scent dispensing device 132 settings, turn scent dispensers 132 on and off, purchase scent vials 250 for the scent dispensing device 132, provide feedback about optimized scent settings, set up a scent dispensing device 132, register an account, view analytics reflecting the user's historical use of his/her scent dispensing device 132, enable user profiles to use and setup scent profiles for the scent dispensing device 132, set a profile hierarchy (e.g., set which user profile(s) is/are the dominant user profile), set motor speeds for the fan 190, manage various scent settings, etc. In some implementations, the scent application may also be able to guide initial scent dispenser 132 installation as described elsewhere herein.

The dispenser management server 150 may include one or more computing devices having data processing, storing, and communication capabilities. For example, the server 150 may include one or more hardware servers, virtual servers, server arrays, storage devices and/or systems, etc., and/or may be centralized or distributed/cloud-based. In some embodiments, the server 150 may include one or more virtual servers, which operate in a host server environment and access the physical hardware of the host server including, for example, a processor, memory, storage, network interfaces, etc., via an abstraction layer (e.g., a virtual machine manager).

While not depicted, the server 150 may include a (physical, virtual, etc.) processor, a non-transitory memory, a network interface, and a data store 170, which may be communicatively coupled by a communications bus. Similarly, the client device 106 may include a physical processor, a non-transitory memory, a network interface, a display, an input device, a sensor, and a capture device. It should be understood that the server and the client device may take other forms and include additional or fewer components without departing from the scope of the present disclosure.

Software operating on the server 150 (e.g., the dispenser installation application 160, an operating system, device drivers, etc.) may cooperate and communicate via a software communication mechanism implemented in association with a server bus. The software communication mechanism can include and/or facilitate, for example, inter-process communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

As shown, the server 150 may include a dispenser installation application 160 embodying a remotely accessible scent service. The dispenser installation application 160 may send data to and receive data from the other entities of the system including the controllers 188, the mobile device(s) 106, etc. The dispenser installation application 160 may be configured to store and retrieve data from one or more information sources, such as the data store 170. In addition, while a single server 150 is depicted in FIG. 1, it should be understood that one or more servers 150 may be included.

In some embodiments, the dispenser firmware 194, the scent application, the dispenser installation application 160, the sensor mapping application 164, etc., may require users to be registered to access the acts and/or functionality provided by them. For example, to access various acts and/or functionality provided by the scent application, dispenser installation application 160, and/or scent dispensers 132, these components may require a user to authenticate his/her identity (e.g., by confirming a valid electronic address). In some instances, these entities 132, 150, etc., may interact with a federated identity server (not shown) to register/authenticate users. Once registered, these entities 132, 150, etc., may require a user seeking access to authenticate by inputting credentials in an associated user interface.

FIG. 2 is a block diagram illustrating the example client device 106. As depicted, the client device 106 may include a processor 202, memory 204, communication unit 206, sensor mapping application 164b, device sensor(s) 212, and a storage device 243 which are communicatively coupled by a communications bus 220. However, it should be understood that the client device 106 is not limited to such and may include other elements, including, for example, a display (not shown) and/or an input device (not shown).

The processor 202 may execute software instructions by performing various input/output, logical, and/or mathematical operations. The processor 202 has various computing architectures to process data signals including, for example, a complex instruction set computer (CISC) architecture, a reduced instruction set computer (RISC) architecture, and/or an architecture implementing a combination of instruction sets. The processor 202 may be physical and/or virtual, and may include a single core or plurality of processing units and/or cores.

The memory 204 is a non-transitory computer-readable medium that is configured to store and provide access to data to the other elements of the client device 106. In some implementations, the memory 204 may store instructions and/or data that may be executed by the processor 202. For example, the memory 204 may store the location application 210. The memory 204 is also capable of storing other instructions and data, including, for example, an operating system, hardware drivers, other software applications, data, etc. The memory 204 may be coupled to the bus 220 for communication with the processor 202 and the other elements of the client device 106.

The communication unit 206 may include one or more interface devices (I/F) for wired and/or wireless connectivity with the network 102 and/or other devices. In some implementations, the communication unit 206 may include transceivers for sending and receiving wireless signals. For instance, the communication unit 206 may include radio transceivers for communication with the network 102 and for communication with nearby devices using close-proximity (e.g., Bluetooth®, NFC, etc.) connectivity. In some implementations, the communication unit 206 may include ports for wired connectivity with other devices. For example, the communication unit 206 may include a CAT-5 interface, Thunderbolt™ interface, FireWire™ interface, USB interface, etc. In further implementations, the Communication Unit 206 may include one or more ultrawideband Bluetooth antenna that can transmit and receive signals to other Bluetooth enabled devices.

In some implementations, the client device 106 may include a display (not shown) that may display electronic images and data output by the client device 106 for presentation to a user 112. The display may include any conventional display device, monitor or screen, including, for example, an organic light-emitting diode (OLED) display, a liquid crystal display (LCD), etc. In some implementations, the display may be a touch-screen display capable of receiving input from one or more fingers of a user 112. For example, the display may be a capacitive touch-screen display capable of detecting and interpreting multiple points of contact with the display surface. In some implementations, the client device 106 may include a graphics adapter (not shown) for rendering and outputting the images and data for presentation on display. The graphics adapter (not shown) may be a separate processing device including a separate processor and memory (not shown) or may be integrated with the processor 202 and memory 204. In some implementations, the display may include notifications or instructions for a user based on a mapping of a space using one or more device sensor(s) 212.

In some implementations, the client device 106 may include an input device (not shown) that may include any device for inputting information into the client device 106. In some implementations, the input device may include one or more peripheral devices. For example, the input device may include a keyboard (e.g., a QWERTY keyboard), a pointing device (e.g., a mouse or touchpad), microphone, a camera, etc. In some implementations, the input device may include a touch-screen display capable of receiving input from the one or more fingers of the user 112. For instance, the functionality of the input device and the display may be integrated, and a user 112 of the client device 106 may interact with the client device 106 by contacting a surface of the display using one or more fingers. In this example, the user 112 could interact with an emulated (i.e., virtual or soft) keyboard displayed on the touch-screen display by using fingers to contact the display in the keyboard regions.

The sensor mapping application 164b may include software and/or logic for mapping out a space based on captured device sensor data and determining installation locations based on the mapping as described above in FIG. 1. In some implementations, the sensor mapping application 164b may process the captured data in order to perform a mapping of at least a portion of a space. In further implementations, the sensor mapping application 164b may process the captured data from the device sensor(s) 212 in order to determine which data points may be sent to the dispenser installation application 160 for mapping.

The device sensor(s) 212 may include various sensors that are capable of capturing data about a space that can be used for mapping the space. Non-limiting example device sensor(s) cameras, microphones and audio outputs that can measure soundwave distance, Lidar, lasers, radar, RFID sensors, radio beacons, ultrasound identification, proximity sensors, and/or other sensor mapping devices that can be positioned and/or installed in common client devices 106. These various device sensor(s) 212 can capture device sensor data that can be used to map out the space. In some implementations, these device sensor(s) 212 may be configured to interact with separate sensor(s) 186 on the scent dispensing device 132, such as sound waves or wifi signals that can determine a distance or other spatial inference between the device sensor(s) 212 and the separate sensor(s) 186. Using this communication between the sensors 186 and device sensors 212, more exact dimensions can be determined for a room space when those communication data between the two sensors is provided to the sensor mapping application 164.

The storage device 243 is an information source for storing and providing access to stored data, user profile information, object data, calibration data, and/or any other information generated, stored, and/or retrieved by the sensor mapping application 164b. In some implementations, some of the location data gathered by the device sensor(s) 212 may be stored on the storage device 243 before being sent to the dispenser installation application 160.

In some implementations, the storage device 243 may be included in the memory 204 or another storage device coupled to the bus 220. In some implementations, the storage device 243 may be or included in a distributed data store, such as a cloud-based computing and/or data storage system. In some implementations, the storage device 243 may include a database management system (DBMS). For example, the DBMS could be a structured query language (SQL) DBMS. For instance, storage device 243 may store data in an object-based data store or multi-dimensional tables comprised of rows and columns, and may manipulate, i.e., insert, query, update, and/or delete, data entries stored in a verification data store using programmatic operations (e.g., SQL queries and statements or a similar database manipulation library). Additional characteristics, structure, acts, and functionality of the storage device 243 is discussed elsewhere herein.

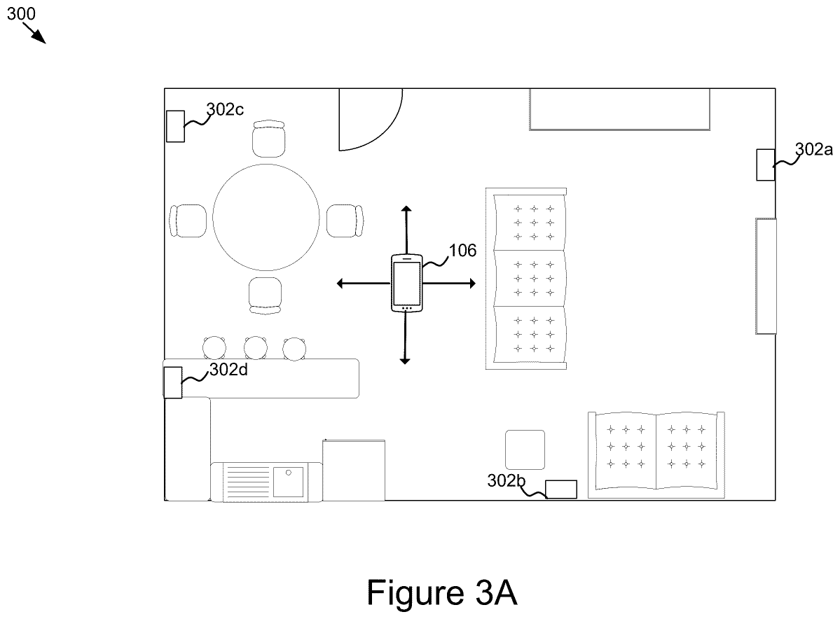

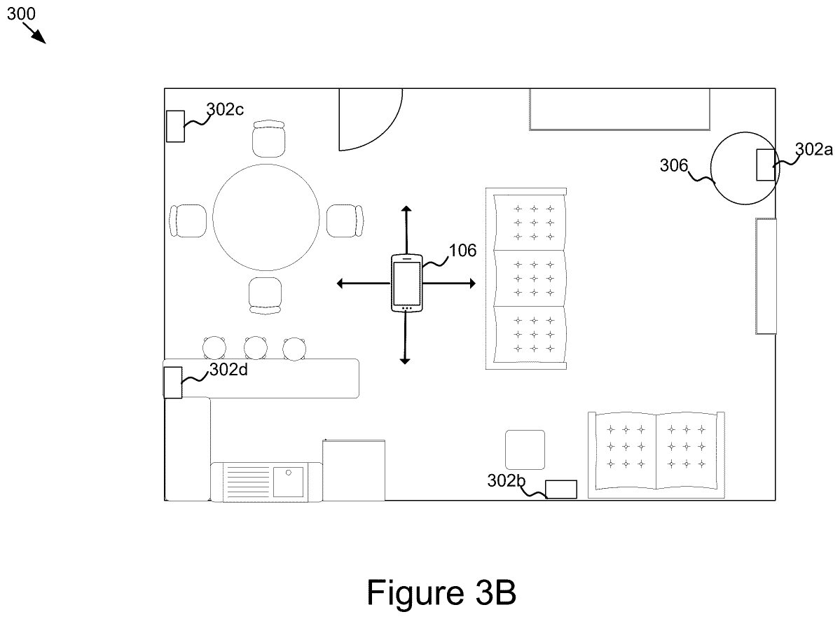

FIGS. 3A and 3B depict a graphical representation of using device sensors to install a scent dispensing device 132 in a space 300. As shown in FIG. 3A, the client device 106 may present instructions to a user for installing a scent dispensing device 132. The instructions may include accessing one or more device sensor(s) 212, such as a camera to take pictures of a space and/or a lidar sensor to map a space. A user 112 (not shown) can move the client device 106 as instructed by the sensor mapping application 164 to capture a space for mapping, such as by rotating the camera of the client device 106 to view the room, or other types of captured sensor data. The space 300 may include one or more scent dispensing device 132 installation locations 302, such as the four scent dispensing applications 302a-302d shown in FIG. 3A. As the user moves the client device 106 around the space 300 and the device sensor(s) 212 capture data about the space 300, the sensor mapping application 164 can determine the one or more installation locations 302 and may determine which of those installation locations 302 are optimal for installation of a scent dispensing device 132. The sensor mapping application 164 can used the captured data bout the space 300 to create a space mapping that maps out various objects included in the space mapping, such as obstructions, like furniture, couches, walls, tables, cubicles, etc., windows and/or doors that may affect airflow paths through the space, walkways where people are likely to move in the space to disperse air, etc., heating/cooling vents and intakes that may adjust various airflow patterns in a room and affect scent disbursement, power outlet locations, clear areas on surfaces such as tables, counters, desks, where battery powered scent dispensing devices 132 may be positioned, and/or other context that may inform the sensor mapping application 164 about how installing a scent dispensing device 132 at a specific location will affect the scent dispensing experience, such as through airflow patterns, scent dispersal through the space, etc. In some implementations, the sensor mapping application 164 can determine various relative positions for the various objects in the space mapping, such as distances between a blocking object and/or a scent dispensing device relative to power outlet or other installation location, etc. In some implementations, these relative positions may further be updated to include dimensions or distances between objects relative to other objects in the space mapping.

As shown in FIG. 3B, the sensor mapping application 164 may be able to determine the four installation locations 302a-302d, such as based on proximity to power outlets, clear space for scent diffusion, away from airflow issues, such as open doors/windows, etc. As shown in FIG. 3B, the sensor mapping application 164 can determine the optimal installation location 302a as denoted by the circle 306. The sensor mapping application 164 can determine that the optimal installation location 302a is away from blocking objects, such as furniture, as well as being close to an outlet and away from high traffic areas.

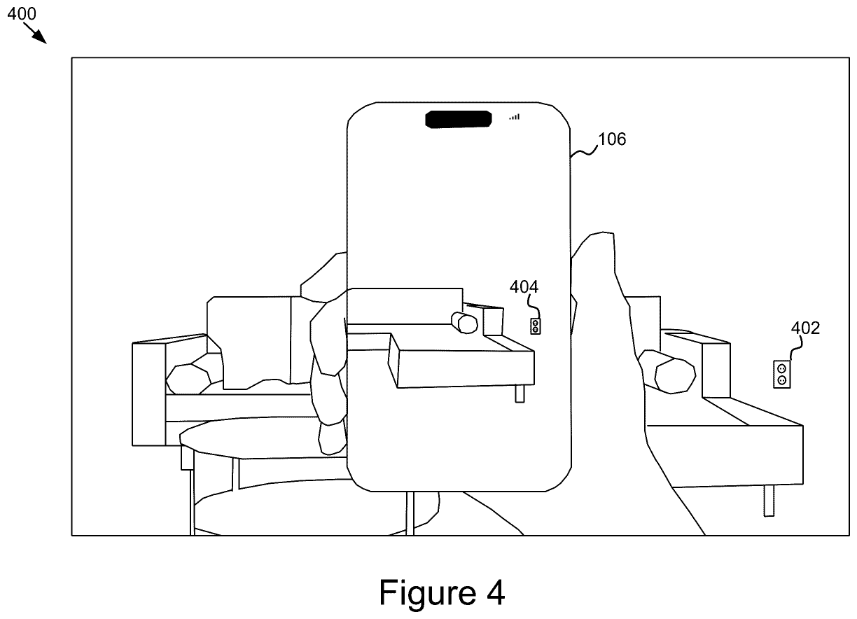

FIG. 4 is a graphical representation 400 depicting using a client device 106 to determine installation locations. As shown in FIG. 4, a device sensor 212 (not shown) is being used to capture and generate a graphical representation of a space. As shown in the example in FIG. 4, a portion of a wall including a couch and an outlet 402 has been captured by the device sensors 212 and a generated image of the wall, couch, and outlet 404 has been depicted in the graphical representation of the display of the client device 106. In some implementations, the client device 106 may also include additional instructions guiding a user 112 in exactly where in the space to install the scent dispensing device 132, such as by overlaying a generated image of the scent dispenser 132 into the generated image on the client device 106 to create an augmented reality instruction, etc. In other implementations, the sensor mapping application 164 may determine one or more optimal installation locations and may guide the user in which installation locations to use.

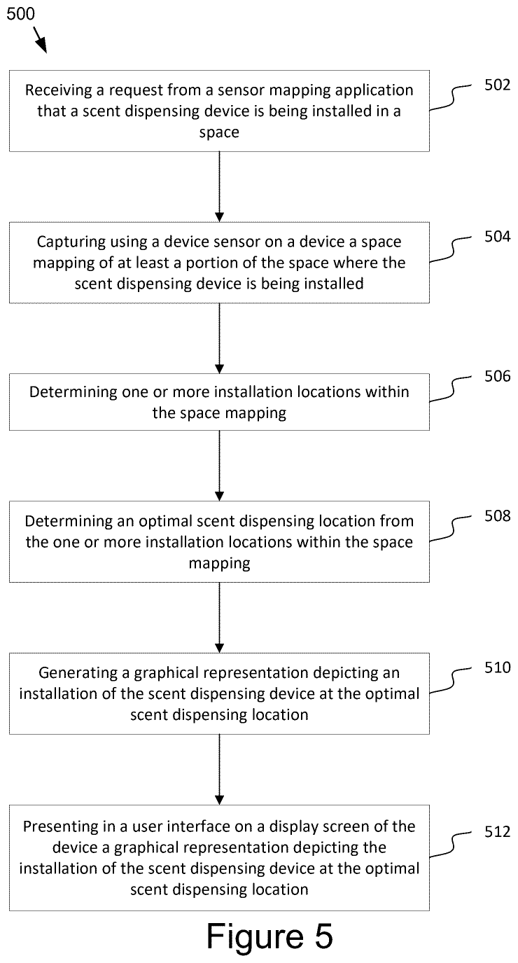

FIG. 5 is a flowchart of an example method 500 of using device sensors to install a scent dispensing device. At step 502, the client device 106 can receive a request from a sensor mapping application 164 that a scent dispensing device 132 is being installed in a space. The request can be triggered, such as by a client device 106 running a scent application during initial installation of a scent dispensing device 132. In some implementations, an event trigger can be sent to a server 102 when a scent dispensing device 132 is initially activated that causes a scent application to begin running on a client device 106.

At step 504, the sensor mapping application 164 can begin capturing, using a device sensor 212, a space mapping of at least a portion of the space where the scent dispensing device 132 is being installed. The device sensor 212 may be a lidar sensor that maps out the space using various cameras and/or a camera that captures the space and performs image detection of the captured images to determine the space mapping. The lidar sensor may send out a plurality of pulses of light and determine distances between the lidar sensor and objects the light reflects off of in order to create/generate/determine a space mapping of distances between various objects. In some implementations, the sensor mapping application 164 can use artificial intelligence to assist in the space mapping and infer details, such as measurements, object recognition, objects used for scent dispensing device 132 installation, such as power outlets, etc.

At step 506, the sensor mapping application 164 can determine one or more installation locations within the space mapping. In some implementations, the installation locations can be locations proximate to power outlets when a scent dispensing device 132 requires a power outlet to operate. In other implementations, such as where a battery powered scent dispensing device 132 is being used, the sensor mapping application 164 can determine open areas away from objects that could block scent diffusion or away from high traffic areas with disruptive airflows, etc. In some implementations, the sensor mapping application 164 can use machine learning algorithms to determine installation locations and may learn and update the process of determining the installation locations over time as the installation locations are identified. In some implementations, the sensor mapping application 164 can determine a size and/or area of a space and can provide recommendations, such as a quantity of scent dispensing devices 132 to install or a specific type of scent dispensing device 132, such as if there are different types of scent dispensing devices 132 can disperse scents differently, such as large scent dispensing devices 132 configured for large spaces and small scent dispensing devices 132 configured for smaller spaces, etc.

At step 508, the sensor mapping application 164 can determine an optimal scent dispensing location from the one or more installation locations within the space mapping. In some implementations, determining the optimal scent dispensing location may include comparing and scoring each of the determined installation locations based on predetermined criteria, such as open space around the location, proximity to high traffic areas, amount of space the scent would be able to diffuse into from the location, proximity to power outlets, proximity to other scent dispensing devices 132, etc. Based on the scores for each of the installation locations, an optimal scent dispensing location may be determined based on the generated scores or rankings.

At step 510, the sensor mapping application 164 may generate a graphical representation depicting an installation of the scent dispensing device at the optimal scent dispensing location. As described elsewhere herein, the sensor mapping application 164 may generate one or more guides that can be displayed as overlays on a mapping of a space to show a user where to position and/or install a scent dispensing device 132. At step 512, the sensor mapping application 164 can present in a user interface on a display screen of the client device 106 a graphical representation depicting the installation of the scent dispensing device 132 at the optimal scent dispensing location. The graphical representation may depict to a user 112 with easy-to-follow graphics exactly where in their personal space the scent dispensing device 132 can be installed.

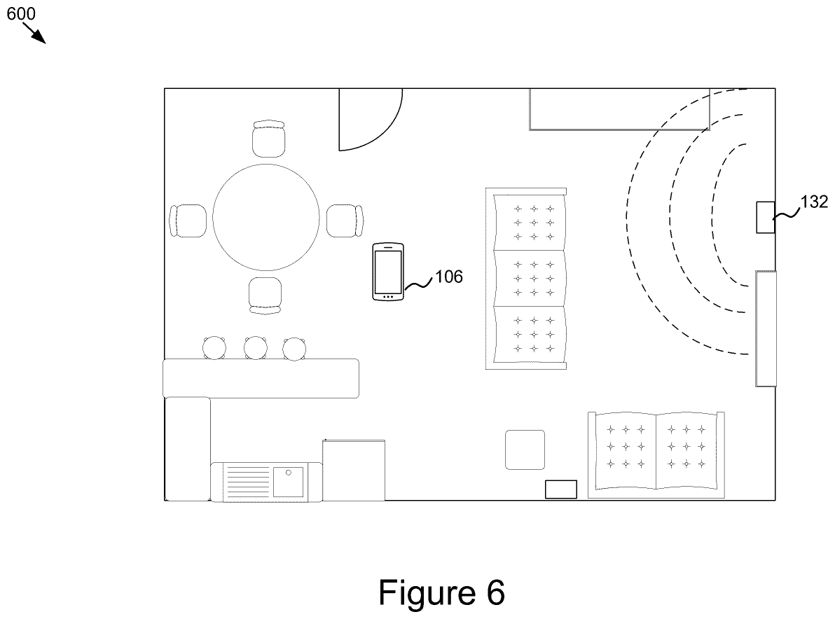

FIG. 6 is a graphical representation 600 of using sound waves to determine scent dispenser installation. As shown in FIG. 6, a scent dispensing device 132 may be positioned at a point in a room and a user may be using a client device 106 to setup the scent dispensing device 132. As part of the setup process, the client device 106 may display a prompt, such as in a user interface, asking for the dimensions of the room that the scent dispensing device 132 is being positioned in. A user may have the option of using sound waves to estimate a size of the room during installation. For example, the client device 106 may be wirelessly connected to the scent dispensing device 132 as described elsewhere herein. The scent dispensing device 132 may emit a series of sounds/sound waves and the client device 106 may use a microphone or other sensor to capture those emitting sounds/sound waves and based on the connection between the two devices, determine how long it took for the sound/sound waves to be detected. Based on the time, the client device 106 using various algorithms, such as in some implementations, various artificial intelligence algorithms, to estimate a distance that the sound wave traveled based on the time it took for the sound/sound waves to be detected. The client device 106 may then inform the user of the estimated distance from the scent dispensing device 132. The client device 106 may further guide the user to move around the space and measure distances from various points using emitted sound waves in order to determine various dimensions of the room using the sound waves. In further implementations, the scent dispensing device 132 may also have sound sensors and may detect a sound emitted from the client device 106 and/or other scent dispensing devices 106 in order to map out various dimensions of the room. In further implementations, the devices may be able to detect various echoes to capture positions of walls and/or various large furniture to use in the mapping. In further implementation, these estimated distances captured using sound may be combined with an image, a video, and/or a lidar mapping to further map out the space in the room for scent dispensing device 132 installation.

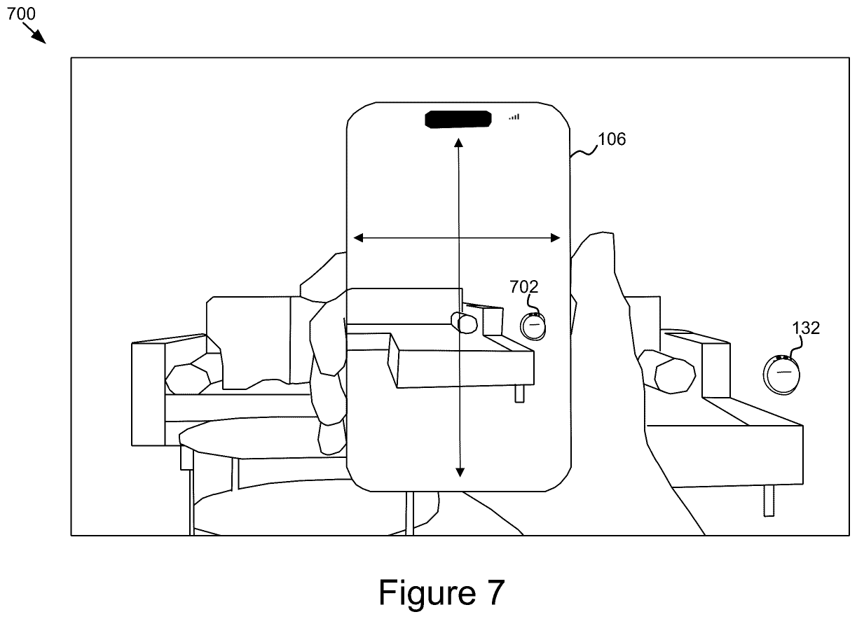

FIG. 7 is a graphical representation 700 of using images to determine scent dispenser installation. As shown in FIG. 7, a user may use a client device 106 to capture one or more images of a space. The client device 106 and/or the sensor mapping application 164 may user various machine learning algorithms, such as artificial intelligence, to estimate a room dimension of the room based on the captured image. The estimated dimension may be a length of the room, a width of the room, a height of the room, a volume of the room, an area of a space in the room, etc. In some implementations, the sensor mapping application 164 may estimate various known sizes for objects in the room in order to estimate the room dimensions. For example, based on a common dimension of a chair or other item, the sensor mapping application 164 may determine the room dimensions. In some implementations, the sensor mapping application 164 may combine various images of the room together in order to provide a more accurate estimate of the room dimensions, such as images of each of the four walls in a square room that can be combined based on common portions of each image in order to provide an overall room dimension, such as an area of the room, etc. In some implementations, the sensor mapping application 164 may provide prompts to a user on the client device 106 in order to guide the user in capture various images and/or video of the room for estimating room dimensions.

In some implementations, during installation of a scent dispensing device 132, the images may include the scent dispensing device 132, that can be used as a scale item 702 for the sensor mapping application 164 to more accurately determine room dimensions. For example, the sensor mapping application 164 may receive various dimensions of different scent dispensing devices 132 and may be able to identify the scent dispensing devices 132 present in images of the room. The sensor mapping application 164 may then use the scale item 702 as the known scale of the scent dispensing device 132 in order to more accurately estimate the room dimensions. This is especially convenient for a user that is installing the scent dispensing device 132, as they can plug the scent dispensing device 132 into the wall and then take a couple of photos of the room that are provided to the sensor mapping application 164. The sensor mapping application 164 may then use those image and the know scale of the plugged in scent dispensing device 132 to estimate dimensions of the room and provide various recommendations to the user, such as scent dispensing device 132 placement recommendations, intensity setting levels to fill the space, etc. This provides a convenient way for a user to quickly be guided through various aspects of installation beyond just having the user plug the scent dispensing device 132 into an outlet.

The foregoing description, for purpose of explanation, has been described with reference to various embodiments and examples. However, the illustrative discussions above are not intended to be exhaustive or to limit the invention to the precise forms disclosed. Many modifications and variations are possible in view of the above teachings. The various embodiments and examples were chosen and described in order to best explain the principles of the invention and its practical applications, to thereby enable others skilled in the art to utilize the innovative technology with various modifications as may be suited to the particular use contemplated. For instance, it should be understood that the technology described herein can be practiced without these specific details in some cases. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms such as “processing,” “computing,” “calculating,” “determining,” “displaying,” or the like, refer to the action and methods of a computer system that manipulates and transforms data represented as physical (electronic) quantities within the computer system's registers and memories into other data similarly represented as physical quantities within the computer system memories or registers or other such information storage, transmission or display devices.

A data processing system suitable for storing and/or executing program code, such as the computing system and/or devices discussed herein, may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices can be coupled to the system either directly or through intervening I/O controllers. The data processing system may include an apparatus may be specially constructed for the required purposes, or it may comprise a general-purpose computer selectively activated or reconfigured by a computer program stored in the computer.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects may not be mandatory or significant, and the mechanisms that implement the specification or its features may have different names, divisions, and/or formats.

Furthermore, the modules, routines, features, attributes, methodologies and other aspects of the disclosure can be implemented as software, hardware, firmware, or any combination of the foregoing. The technology can also take the form of a computer program product accessible from a computer-usable or computer-readable medium providing program code for use by or in connection with a computer or any instruction execution system. Wherever a component, an example of which is a module or engine, of the specification is implemented as software, the component can be implemented as a standalone program, as part of a larger program, as a plurality of separate programs, as a statically or dynamically linked library, as a kernel loadable module, as firmware, as resident software, as microcode, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A method of using device sensors to install a scent dispensing device, comprising:
receiving a request from a sensor mapping application that a scent dispensing device is being installed in a space;
capturing using a device sensor on a device a space mapping of at least a portion of the space where the scent dispensing device is being installed;
determining one or more installation locations within the space mapping;
determining an optimal scent dispensing location from the one or more installation locations within the space mapping;
generating a graphical representation depicting an installation of the scent dispensing device at the optimal scent dispensing location; and
presenting in a user interface on a display screen of the device the graphical representation depicting the installation of the scent dispensing device at the optimal scent dispensing location.

2. The method of claim 1, wherein capturing the space mapping further comprises: determining based on the space mapping one or more dimensions of the space.

3. The method of claim 2, wherein the dimensions of the space includes one or more of a length of the space, a width of the space, a height of the space, a volume of the space, and an area of the space.

4. The method of claim 1, wherein the device sensor is a camera of a client device and the space mapping is one or more images of the space captured by the camera.

5. The method of claim 4, wherein determining the installation locations within the space mapping further comprises performing object recognition to determine one or more objects present within the one or more images.

6. The method of claim 5, wherein the one or more determined objects present within the images may be compared to a database of common objects to determine one or more dimensions of the space.

7. The method of claim 5, wherein determining the installation locations within the space mapping further comprises determining one or more power outlet locations that the scent dispensing device can plug into.

8. The method of claim 7, wherein determining the optimal scent dispensing location from the one or more installation locations within the space mapping further comprises:
determining an obstruction present within the space mapping;
determining a walkway present within the space mapping; and
identifying which of the power outlet locations is optimized based on the determined obstruction and the determined walkway.

9. The method of claim 1, wherein the graphical representation depicting the installation of the scent dispensing device at the optimal scent dispensing location is an overlay and presenting in the user interface on the display screen of the device further comprises placing the overlay over the space mapping on the display screen of the device.

10. The method of claim 1, wherein determining one or more installation locations within the space mapping further comprises:
detecting a scent dispensing device within the space mapping;
estimating space dimensions using a known scale of the scent dispensing device; and
determining the one or more installation locations based on the estimated space dimensions.

11. A scent dispensing device installation system comprising:
a scent dispensing device configured to be installed in a space;
a device sensor configured to capture a space mapping of the space; and
a sensor mapping application configured to:
receive the space mapping from the device sensor;
determine an installation location for the scent dispensing device from the space mapping;
optimize the installation location based on other context in the space mapping; and causing a user interface on a display screen to present a graphical representation depicting the installation of the scent dispensing device at the optimal scent dispensing location.

12. The scent dispensing device installation system of claim 11, wherein when the sensor mapping application receives the space mapping from the device sensor, the sensor mapping application is further configured to determine a dimension of the space based on the space mapping.

13. The scent dispensing device installation system of claim 12, wherein the dimension of the space includes one or more of a length of the space, a width of the space, a height of the space, a volume of the space, and an area of the space.

14. The scent dispensing device installation system of claim 13, wherein the sensor mapping application is further configured to identify an object in the space mapping using image recognition.

15. The scent dispensing device installation system of claim 14, wherein the object in the space mapping is the scent dispensing device and wherein determining the dimensions of the space further comprises using a known scale of the scent dispensing device to estimate the dimensions of the space.

16. The scent dispensing device installation system of claim 11, wherein the sensor device is a camera of a client device and the space mapping is one or more images of the space captured by the camera.

17. The scent dispensing device installation system of claim 11, wherein the sensor device is a lidar sensor that maps out a plurality of distances to capture the space mapping.

18. The scent dispensing device installation system of claim 11, wherein scent dispensing device includes an audio output device and the sensor device includes an audio input device configured to capture a sound emitted from the audio output device and determine a distance between the scent dispensing device and the audio input device to capture the space mapping.

19. A method of guiding installation of a scent dispensing device, the method comprising:
capturing, using a device sensor, a space mapping of a space in which a scent dispensing device is being installed;
identifying, using a processor of a sensor mapping application, one or more objects within the space mapping, the one or more objects including the scent dispensing device, a power outlet, and a blocking object;
determining, using the processor of the sensor mapping application, one or more dimensions of the space using the space mapping;
determining, using the processor of the sensor mapping application, a first relative positions in the space mapping of the power outlet and a second relative position in the space mapping of the blocking object based on the one or more dimensions of the space mapping;
determining, using the processor of the sensor mapping application, an optimal scent dispensing location based on the first relative positions in the space mapping of the power outlet and the second relative position in the space mapping of the blocking object; and
presenting for display in a graphical user interface, an installation instruction to install the scent dispensing device at the optimal scent dispensing location.

20. The method of claim 19, wherein the one or more dimensions of the space include a length of the space, a width of the space, a height of the space, a volume of the space, and an area of the space.
